(19) United States
(12) Patent Application Publication
(10) Pub. No.: US 2021/0209775 A1
(43) Pub. Date: Jul. 8, 2021

(54) Image Processing Method and Apparatus, and Computer Readable Storage Medium

(71) Applicant: Shanghai SenseTime Intelligent Technology Co., Ltd., Shanghai (CN)

(72) Inventor: Tao Song, Shanghai (CN)

(73) Assignee: Shanghai SenseTime Intelligent Technology Co., Ltd., Shanghai (CN)

(21) Appl. No.: 17/210,021

(22) Filed: Mar. 23, 2021

Related U.S. Application Data
(63) Continuation of application No. PCT/CN2019/114563, filed on Oct. 31, 2019.

(30) Foreign Application Priority Data
Dec. 19, 2018 (CN) 201811559600.6

(51) Int. Cl. G06T 7/33; G06T 5/50; G06T 7/00; G06N 3/04; G06T 7/38

(52) U.S. Cl. CPC G06T 7/337; G06T 5/50; G06T 7/0014; G06T 2207/20084; G06T 7/38; G06T 2207/20081; G06N 3/04

(57) ABSTRACT

Embodiments of the present disclosure disclose an image processing method and apparatus, an electronic device, and a computer readable storage medium. The method includes: obtaining an image to be registered and a reference image used for registration; inputting the image to be registered and the reference image into a preset neural network model, where the preset neural network model is obtained by training based on mutual information loss of a preset image to be registered and a preset reference image; and registering the image to be registered with the reference image based on the preset neural network model to obtain a registration result. The precision and real-time performance of image registration can be improved.

### CROSS REFERENCE TO RELATED APPLICATIONS

The present disclosure is a continuation of and claims priority under 35 U.S.C. 120 to PCT Application. No. PCT/CN2019/114563, filed on Oct. 31, 2019, which claims priority to Chinese Patent Application No. 201811559600.6, filed with the Chinese Patent Office on Dec. 19, 2018 and entitled “IMAGE PROCESSING METHOD AND APPARATUS, ELECTRONIC DEVICE, AND COMPUTER READABLE STORAGE MEDIUM”. All the above-referenced priority documents are incorporated herein by reference in their entireties.

### TECHNICAL FIELD

The present disclosure relates to the field of computer vision technologies, and in particular, to an image processing method and apparatus, an electronic device, and a computer readable storage medium.

### BACKGROUND

Image registration is a process of registering two or more images of the same scene or the same target under different acquisition time, different sensors, and different conditions, and is widely applied to a medial image processing process. Medical image registration is an important technology in the field of medical image processing, and plays a more and more important role in clinical diagnosis and treatment.

Modern medicine often requires comprehensive analysis of medical images obtained from multiple modalities or multiple time points, so several images need to be registered before analysis. A traditional deformable registration method is a process of calculating a similarity between a registered image and a reference image by means of a similarity measure function by continuously calculating one correspondence of each pixel, and continuously performing iteration until reaching an appropriate result.

### SUMMARY

Embodiments of the present disclosure provide technical solutions of image processing.

A first aspect of the embodiments of the present disclosure provides an image processing method, including:

obtaining an image to be registered and a reference image used for registration;

inputting the image to be registered and the reference image into a preset neural network model, where the preset neural network model is obtained by training based on mutual information loss of a preset image to be registered and a preset reference image; and

registering the image to be registered with the reference image based on the preset neural network model to obtain a registration result.

In one optional implementation, before obtaining the image to be registered and the reference image used for registration, the method further includes:

obtaining an original image to be registered and an original reference image, and performing image normalization processing on the original image to be registered and the original reference image to obtain the image to be registered and the reference image that meet target parameters. In this way, unrelated information in the image is eliminated, useful and real information is restored, the detectability of related information is enhanced, and data is maximally simplified, so that the reliability of feature extraction and image segmentation, matching and recognition is improved.In one optional implementation, performing image normalization processing on the original image to be registered and the original reference image to obtain the image to be registered and the reference image that meet the target parameters includes:

converting the original image to be registered into the image to be registered within a preset gray value range and with a preset image size; and

converting the original reference image into the reference image within the preset gray value range and with the preset image size. In this way, subsequent image processing process is more accurate and stable.

In one optional implementation, the preset neural network model includes a registering model and a mutual information estimating network model, and a training process of the preset neural network model includes:

obtaining the preset image to be registered and the preset reference image, and inputting the preset image to be registered and the preset reference image into the registering model to generate a deformable field;

estimating, in a process of registering with the preset reference image based on the deformable field and the preset image to be registered, mutual information of a registered image and the preset reference image by means of the mutual information estimating network model to obtain the mutual information loss; and

performing parameter updating on the registering model and the mutual information estimating network model based on the mutual information loss to obtain a trained preset neural network model. In this way, the precision and the real-time performance of image registration are improved by registering the image to be registered with the reference image based on the preset neural network model to obtain the registration result.

In one optional implementation, estimating the mutual information of the registered image and the preset reference image by means of the mutual information estimating network model to obtain the mutual information loss includes:

obtaining joint probability distribution and marginal probability distribution by means of the mutual information estimating network model based on the registered image and the preset reference image; and

calculating the mutual information loss according to the joint probability distribution and the marginal probability distribution. In this way, adversarial training of a generating model is enhanced, and the bottleneck of a supervised learning classification task is broken through.

In one optional implementation, performing parameter updating on the registering model and the mutual information estimating network model based on the mutual information loss to obtain the trained preset neural network model includes:

performing parameter updating of a first threshold number of times on the registering model based on the mutual information loss, and performing parameter updating of a second threshold number of times on the mutual information estimating network model based on the mutual information loss, to obtain the trained preset neural network model. In this way, the parameters of the registering model and the mutual information estimating network model are continuously updated to guide and complete the training of the two networks.

In one optional implementation, the method further includes:

performing parameter updating of a preset learning rate and a third threshold number of times on the preset neural network model based on a preset optimizer. In this way, a final trained preset neural network model is obtained.

In one optional implementation, after obtaining the preset image to be registered and the preset reference image, the method further includes:

performing image normalization processing on the preset image to be registered and the preset reference image to obtain the preset image to be registered and the preset reference image that meet preset training parameters; and

inputting the preset image to be registered and the preset reference image into the registering model to generate the deformable field includes:

inputting the preset image to be registered and the preset reference image that meet the preset training parameters into the registering model to generate the deformable field.

Here, the normalization processing is to facilitate subsequent loss calculation without causing gradient explosion.

A second aspect of the embodiments of the present disclosure provides an image processing apparatus, including an obtaining module and a registering module, where:

the obtaining module is configured to obtain an image to be registered and a reference image used for registration;

the registering module is configured to input the image to be registered and the reference image into the preset neural network model, where the preset neural network model is obtained by training based on the mutual information loss of the preset image to be registered and the preset reference image; and

the registering module is further configured to register the image to be registered with the reference image based on the preset neural network model, to obtain the registration result.

In one optional implementation, the image processing apparatus further includes:

a preprocessing module, configured to obtain the original image to be registered and the original reference image, and perform image normalization processing on the original image to be registered and the original reference image to obtain the image to be registered and the reference image that meet the target parameters.

In one optional implementation, the preprocessing module is specifically configured to:

convert the original image to be registered into the image to be registered within the preset gray value range and with the preset image size; and convert the original reference image into the reference image within the preset gray value range and with the preset image size.

In one optional implementation, the preset neural network model includes a registering model and a mutual information estimating network model, and the registering module includes a registering unit, a mutual information estimating unit, and an updating unit, where:

the registering unit is configured to obtain the preset image to be registered and the preset reference image, and input the preset image to be registered and the preset reference image into the registering model to generate a deformable field;

the mutual information estimating unit is configured to estimate, in a process of registering with the preset reference image by the registering module based on the deformable field and the preset image to be registered, mutual information of a registered image and the preset reference image by means of the mutual information estimating network model to obtain the mutual information loss; and

the updating unit is configured to perform parameter updating on the registering model and the mutual information estimating network model based on the mutual information loss to obtain a trained preset neural network model.

In one optional implementation, the mutual information estimating unit is specifically configured to:

obtain the joint probability distribution and the marginal probability distribution by means of the mutual information estimating network model based on the registered image and the preset reference image; and calculate the mutual information loss according to the joint probability distribution and the marginal probability distribution.

In one optional implementation, the updating unit is specifically configured to:

perform parameter updating of the first threshold number of times on the registering model based on the mutual information loss, and perform parameter updating of the second threshold number of times on the mutual information estimating network model based on the mutual information loss, to obtain the trained preset neural network model.

In one optional implementation, the updating unit is further configured to perform parameter updating of a preset learning rate and a third threshold number of times on the preset neural network model based on a preset optimizer.

In one optional implementation, the preprocessing module is further configured to:

perform, after obtaining the preset image to be registered and the preset reference image, image normalization processing on the preset image to be registered and the preset reference image to obtain the preset image to be registered and the preset reference image that meet preset training parameters; and

the registering module is further configured to input the preset image to be registered and the preset reference image that meet the preset training parameters into the registering model to generate the deformable field.

A third aspect of the embodiments of the present disclosure provides an electronic device, including a processor and a memory, where the memory is configured to store one or more programs; the one or more programs are configured to be executed by the processor; the program is used for executing, for example, some or all of the steps described in any one method in the first aspect of the embodiments of the present disclosure.

A fourth aspect of the embodiments of the present disclosure provides a computer readable storage medium, which is configured to store a computer program for electronic data interchange, where the computer program enables a computer to execute, for example, some or all of the steps described in any one method in the first aspect of the embodiments of the present disclosure.

A fifth aspect of the embodiments of the present disclosure provides a computer program, where the computer program includes a computer readable code, and when the computer readable code runs in an electronic device, a processor in the electronic device executes some or all of the steps described in any one method in the first aspect of the embodiments of the present disclosure.

In the embodiments of the present disclosure, by obtaining an image to be registered and a reference image used for registration, inputting the image to be registered and the reference image into a preset neural network model, where the preset neural network model is obtained by training based on the mutual information loss of the preset image to be registered and the preset reference image, and registering the image to be registered with the reference image based on the preset neural network model to obtain the registration result, the precision and real-time performance of image registration can be improved.

### BRIEF DESCRIPTION OF THE DRAWINGS

To be more clearly describe the technical solutions in the embodiments of the present disclosure or the prior art, the accompanying drawings required to be used in the description of the embodiments or the prior art are simply introduced below.

FIG. 1 is a schematic flowchart of an image processing method according to embodiments of the present disclosure;

FIG. 2 is a schematic flowchart of a training method of a preset neural network according to embodiments of the present disclosure;

FIG. 3 is a schematic structural diagram of an image processing apparatus according to embodiments of the present disclosure; and

FIG. 4 is a schematic structural diagram of another image processing apparatus according to embodiments of the present disclosure.

### DETAILED DESCRIPTION

To make a person skilled in the art have a better understanding of the solutions of the present disclosure, the technical solutions in the embodiments of the present disclosure are hereinafter described clearly and completely with reference to the accompanying drawings in the embodiments of the present disclosure. Apparently, the described embodiments are merely some of embodiments of the present disclosure, rather than all the embodiments. Based on the embodiments in the present disclosure, all other embodiments obtained by a person of ordinary skill in the art without involving an inventive effort shall fall within the scope of protection of the present disclosure.

The terms “first”, “second”, and the like in the description, the claims, and the accompanying drawings in the present disclosure are used for distinguishing different objects, rather than describing specific sequences. In addition, the terms “include” and “have” and any deformation thereof aim at covering non-exclusive inclusion. For example, the process, method, system, product, or device including a series of steps or units is not limited to the listed steps or units, but also optionally includes steps or units that are not listed or other steps or units inherent to the process, method, product, or device.

Reference herein to “embodiment” means that a particular feature, structure, or characteristic described in connection with the embodiment can be included in at least one embodiment of the present disclosure. The appearances of this phrase in various places in the specification are not necessarily all referring to the same embodiment, nor are separate or alternative embodiments mutually exclusive of other embodiments. The embodiments described herein, explicitly and implicitly understood by a person skilled in the art, may be combined with other embodiments.

Multiple other terminal devices may be allowed to access the image processing apparatus involved in the embodiments of the present disclosure. The image processing apparatus may be an electronic device and includes a terminal device. In a specific implementation, the terminal device includes but not limited to other portable devices, such as a mobile phone, a laptop or a tablet computer with a touch-sensitive surface (e.g. a touch screen display and/or a touch pad). It should also be understood that, in some embodiments, the device is not a portable communications device, but is a desktop computer with a touch-sensitive surface (e.g., a touch screen display and/or a touch pad).

The concept of deep learning in the embodiments of the present disclosure originates from research on artificial neural networks. A multi-layer perceptron including a plurality of hidden layers is a deep learning structure. During the deep learning, an attribute type or feature is represented by combining low-layer features to form a more abstract high-layer so as to discover distributed feature representations of data.

Deep learning is a learning method based on characterization of data in machine learning. An observed value (such as an image) may be represented in various ways, such as the vector of the intensity value of each pixel, or more abstractly represented as a series of edges, a region having a particular shape, and the like. Certain specific representation methods may be used to learn a task from instances more easily (such as face recognition or facial expression recognition). The advantage of deep learning is to substitute manual acquisition of features with efficient unsupervised or semi-supervised algorithms for feature learning and hierarchical feature extraction. Deep learning is a new field in machine learning research, and the motivation of the deep learning is to build a neural network that simulates the human brain for analytical learning. It mimics the mechanism of the human brain to interpret data such as an image, a sound and a text.

The embodiments of the present disclosure are introduced in detail below.

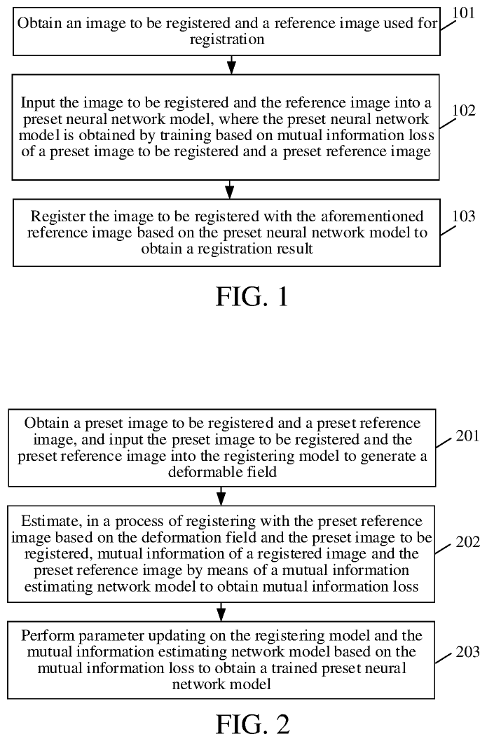

Please refer to FIG. 1. FIG. 1 is a schematic flowchart of an image processing method according to embodiments of the present disclosure. As shown in FIG. 1, the image processing method may be implemented by the image processing apparatus and includes the following steps.

At step 101, an image to be registered and a reference image used for registration are obtained.

Image registration is a process of registering two or more images of the same scene or the same target under different acquisition time, different sensors, and different conditions, and is widely applied to a medial image processing process. Medical image registration is an important technology in the field of medical image processing, and plays a more and more important role in clinical diagnosis and treatment. Modern medicine often requires comprehensive analysis of medical images obtained from multiple modalities or multiple time points, so several images need to be registered before analysis.

The image to be registered (moving) and the reference image used for registration (fixed) mentioned in the embodiments of the present disclosure may both be medical images obtained by means of at least one medical image device, especially for images of some organs that may deform, such as a lung CT image, where the image to be registered and the reference image used for registration generally are images of the same organ collected at different time points or under different conditions.

Because the medical images needing to be registered may have diversity, it may be reflected in the images as the diversity of features such as an image gray value and an image size. Optionally, before step 101, an original image to be registered and an original reference image may be obtained, and image normalization processing is performed on the original image to be registered and the original reference image to obtain the image to be registered and the reference image that meet the target parameters.

The aforementioned target parameters may be understood as parameters describing image features, i.e., prescribed parameters used for making the original image data in a uniform style. For example, the target parameters may include: parameters for describing features such as image resolution, image gray, and the image size.

The original image to be registered may be the medical image obtained by means of at least one medical image device, and in particular, may be the image of an organ that may deform, and has diversity, which is reflected in the image as the diversity of the features such as the image gray value and the image size. Before performing registration, some basic preprocessing may be performed on the original image to be registered and the original reference image, and preprocessing may also be performed only on the original image to be registered. The preprocessing may include the image normalization processing Image preprocessing is mainly aimed at eliminating the unrelated information in the image, restoring the useful and real information, enhancing the detectability of the related information, and maximally simplifying data, thereby improving the reliability of feature extraction and image segmentation, matching and recognition. The image normalization in the embodiments of the present disclosure refers to a process of performing a series of standard processing transformation on the image, so that the image is transformed into a fixed standard form. The standard image is called as a normalized image. By using the invariant moment of the image to find a group of parameters, image normalization may eliminate the influence of other transformation functions on image transformation, and convert an original image to be processed into a corresponding unique standard form. The image in the standard form has invariant features on affine transformation, such as translation, rotation and scaling. Therefore, images in a uniform style may be obtained by means of the image normalization processing, and the stability and accuracy of the subsequent processing are improved.

Specifically, the original image to be registered is converted into the image to be registered within a preset gray value range and with a preset image size.

The original reference image is converted into the reference image within the preset gray value range and with the preset image size.

The aforementioned conversion is mainly aimed at obtaining the image to be registered and the reference image with the same style. That is, it can be understood that the original image to be registered and the original reference image are converted to fall within the same gray value range and have the same image size, and may also be converted to have the same image size or fall with the same gray value range, so that the subsequent image processing process is more accurate and stable.

The image processing apparatus in the embodiments of the present disclosure may store the preset gray value range and the preset image size. Resampling is performed by means of simple ITK software, so that the positions and resolutions of the image to be registered and the reference image need to be substantially consistent. ITK is an open source cross-platform system, and provides a developer with a complete set of software tools for image analysis.

The preset image size may be 416*416*80 (length*width*height), and the sizes of the image to be registered and the reference image are consistent and made to be 416*416*80 by cutting or filing (zero-filling).

The diversity of the original image data is reduced by means of preprocessing, so that the neural network model can provide more stable judgment.

Registration for two medical images 1 and 2 that are obtained at different acquisition times or/and under different conditions is to look for a mapping relationship P, so that each point on the image 1 corresponds to a unique point on the image 2. Moreover, the two points corresponds to the same anatomic position. The mapping relationship P represents a group of continuous spatial transformations. Common spatial geometric transformations include rigid body transformation, affine transformation, projective transformation, and nonlinear transformation.

The rigid body transformation means that a distance and a parallel relationship between any two points in an object remain unchanged. The affine transformation is the simplest non-rigid transformation, and is a transformation that preserves parallelism but does not preserve angles and allows distance changes. Moreover, in many important clinical applications, it is often necessary to apply deformable image registration methods. For example, when studying the image registration of abdominal and thoracic organs, because the position, size and morphology of internal organs and tissues are changed due to physiological movement or the movement of a patient, deformable transformation is required to compensate for image deformation.

In the embodiments of the present disclosure, the aforementioned preprocessing further includes the rigid transformation, i.e., the rigid transformation of the image is first performed, and then the aforementioned image registration is realized according to the method in the embodiments of the present disclosure.

In the field of image processing, only when the position (translation transformation) and orientation (rotation transformation) of the object are changed but the shape is not changed, the obtained transformation is called as the aforementioned rigid transformation.

At step 102, the image to be registered and the reference image are input into the preset neural network model, where the preset neural network model is obtained by training based on mutual information loss of the preset image to be registered and the preset reference image.

In the embodiments of the present disclosure, the image processing apparatus may store the neural network model, and the preset neural network model may be obtained by pre-training.

The neural network model may be obtained by training based on the manner of neuron estimation mutual information, and specifically may be obtained by training based on the mutual information loss of the preset image to be registered and the preset reference image.

The preset neural network model includes a registering model and a mutual information estimating network model. The training process of the preset neural network model includes:

obtaining the preset image to be registered and the preset reference image, and inputting the preset image to be registered and the preset reference image into the registering model to generate a deformable field;

estimating, in a process of registering with the preset reference image based on the deformation field and the preset image to be registered, mutual information of the preset image to be registered and the preset reference image by means of the mutual information estimating network model to obtain the mutual information loss; and

performing parameter updating on the registering model and the mutual information estimating network model based on the mutual information loss to obtain a trained preset neural network model.

For example, the mutual information between high-dimensional continuous random variables may be estimated based on a neural network gradient descent algorithm. For example, a Mutual Information Neural Estimation (MINE) algorithm is linearly measureable in dimension and sample size, and can be trained using a backpropagation algorithm. The MINE algorithm may maximize or minimize the mutual information, enhance the adversarial training of the generating model, and break through the bottleneck of a supervised learning classification task.

At step 103, the image to be registered is registered with the reference image based on the preset neural network model to obtain a registration result.

Image registration generally relates to first performing feature extraction on two images to obtain feature points, then finding matched feature point pairs by performing similarity measurement, obtaining an image spatial coordinate transformation function by means of the matched feature point pairs, and finally performing image registration by means of coordinate transformation parameters.

In the embodiments of the present disclosure, the convolutional layer of the preset neural network model may be 3D convention. The deformable field is generated by means of the preset neural network model, then by means of a 3D spatial conversion layer, deformable transformation is performed on the image to be registered that needs to be deformed to obtain the registration result subsequent to the registration, i.e., including a generated registration result image (moved).

In the preset neural network model, to ensure the smoothness of the deformable field, an L2 loss function is used to constrain the gradient of the deformable field. The mutual information is estimated by means of one neural network and used as the loss function to evaluate the similarity between the registered image and the reference image to guide the training of the network.

In an existing method, supervised deep learning is used to perform registration, there is basically no gold standard, a traditional registration method must be used to obtain a marker, the processing time is long, and the registration precision is limited. In addition, when the traditional method is used to perform registration, it is required to calculate the transformation relationship of each pixel, the calculation amount is huge, and time consumption is also very large.

Unsupervised learning refers to resolving one or more problems in mode recognition according to the training sample of an unknown (unmarked) category. In the embodiments of the present disclosure, the neural network based on unsupervised deep learning is used to perform image registration, and may be used for the registration of any internal organ that may deform. In the embodiments of the present disclosure, a GPU may be used to execute the aforementioned method to obtain the registration result within several seconds, so it is more efficient.

In the embodiments of the present disclosure, by obtaining an image to be registered and a reference image used for registration, inputting the image to be registered and the reference image into a preset neural network model, where the preset neural network model is obtained by training based on the mutual information loss of the preset image to be registered and the preset reference image, and registering the image to be registered with the reference image based on the preset neural network model to obtain the registration result, the precision and real-time performance of image registration can be improved.

Please refer to FIG. 2. FIG. 2 is a schematic flowchart of another image processing method disclosed in the embodiments of the present disclosure, and specifically is a schematic flowchart of a training method of a preset neural network. FIG. 2 is obtained by means of further optimization on the basis of FIG. 1. An execution subject of the steps of the embodiments of the present disclosure may be an image processing apparatus which may be identical to or different from the image processing apparatus in the method in the embodiments shown in FIG. 1. As shown in FIG. 2, the image processing method includes the following steps.

At step 201, a preset image to be registered and a preset reference image are obtained, and the preset image to be registered and the preset reference image are input into the registering model to generate a deformable field.

Similar to the embodiments shown in FIG. 1, the preset image to be registered (moving) and the preset reference image (fixed) may both be medical images obtained by means of various medical image devices, especially for images of the deformable organ, such as the lung CT image, where the image to be registered and the reference image used for registration generally are images of the same organ collected at different time points or under different conditions. The word “preset” here is aimed at distinguishing from the image to be registered and the reference image in the embodiments shown in FIG. 1, and the preset image to be registered and the preset reference image here are mainly used as the inputs of the preset neural network model, and are configured to perform training on the preset neural network model.

Because the medical images needing to be registered may have diversity, it may be reflected in the images as the diversity of features such as an image gray value and an image size. Optionally, after obtaining the preset image to be registered and the preset reference image, the method may also include:

performing image normalization processing on the preset image to be registered and the preset reference image to obtain the preset image to be registered and the preset reference image that meet preset training parameters,

where inputting the preset image to be registered and the preset reference image into the registering model to generate the deformable field includes:

inputting the preset image to be registered and the preset reference image that meet the preset training parameters into the registering model to generate the deformable field.

The preset training parameters may include the preset gray value range and the preset image size (such as 416*416*80). For the process of the image normalization processing, reference may be made to the specific descriptions in step 101 in the embodiments shown in FIG. 1. Optionally, first, the preprocessing performed before registration may include rigid transformation and data normalization. Specifically, resampling is performed by means of simple ITK software, so that the positions and resolutions of the preset image to be registered and the preset reference image need to be substantially consistent. To facilitate operations of a subsequent training process, the image may be cut or filled into a predetermined size. It is assumed that the size of a preset input image is 416*416*80 (length*width*height), it is required to make the sizes of the preset image to be registered and the preset reference image consistent and be 416*416*80 by cutting or filling (zero filling). For important information in the lung CT image, the preset image to be registered and the preset reference image may be normalized to [0, 1] by means of windowing of [4200, 600]. That is, the one greater than 600 in the original image is set to be 1, and the one less than −1200 is set to be 0.

Different organs and tissues have different CT manifestations, i.e., the corresponding gray levels may be different. The so-called windowing is a process of calculating an image by using data obtained by means of a Hounsfield Unit (HU). Different radiodensities correspond to 256 gray values with different degrees. These different gray values may redefine an attenuation value according to different ranges of CT values. It is assumed that the central value of the CT range is not changed, once the defined range becomes narrow, we call as narrow window, a subtle change may be distinguished, which is called as comparative compression in the concept of image processing.

To better extract the important information, the accepted windowing and window may be set on the CT for different tissues in the embodiments of the present disclosure. Herein, the specific values, i.e., −1200 and 600, in the [4200, 600] represent a window, and the range size is 1800, i.e., the windowing. The aforementioned normalization processing is to facilitate subsequent loss calculation without causing gradient explosion.

An L2 loss function may be used, and the characteristic of the L2 loss function is relatively smooth. Here, to respond to an abrupt change caused by the large change of the gradient of the deformable field, and generated winkles and holes, the gradient is represented by means of the difference between adjacent pixels, i.e., to prevent adjacent pixels from changing too much and causing large deformation.

The preset image to be registered and the preset reference image subjected to preprocessing are inputted into a neural network to be trained to generate the deformable field, and then registration with the preset reference image is performed based on the deformable field and the preset image to be registered, i.e., the deformable field and the preset reference image are used to generate the registration result image (moved) after the deformation.

At step 202, in the process of registering with the preset reference image based on the deformable field and the preset image to be registered, mutual information of the registered image and the preset reference image is estimated by means of the mutual information estimating network model to obtain the mutual information loss.

The preset neural network model in the embodiments of the present disclosure may include the mutual information estimating network model and the registering model. The registered image is the image subsequent to registering the preset image to be registered with the preset reference image by means of a registering network this time. In one implementation, by means of the mutual information estimating network model, joint probability distribution and marginal probability distribution may be obtained based on the registered image and the preset reference image; and then the mutual information loss is calculated according to the joint probability distribution and the marginal probability distribution.

For example, the mutual information between high-dimensional continuous random variables may be estimated based on a neural network gradient descent algorithm. For example, a Mutual Information Neural Estimation (MINE) algorithm is linearly measureable in dimension and sample size, and can be trained using a backpropagation algorithm. The MINE algorithm may maximize or minimize the mutual information, enhance the adversarial training of the generating model, and break through the bottleneck of a supervised learning classification task. The mutual information loss may be calculated based on the following mutual information calculation formula (1):

$${I )}_{n} = {sup}_{θ ∈ Θ} {𝔼}_{{P}_{xz}^{( n )}} [ {T}_{θ} ] - log ( {𝔼}_{{P}_{x}^{( n )} ⊗ {\hat{P}}_{z}^{( n )}} [ {e}^{{T}_{θ}} ] ) , \qquad Formula ( 1 )$$

where X and Z may be understood as two input images (i.e., the registered image and the preset reference image); here, X and Z may be understood as a solution space that refers to a vector space consisting of the set of the solutions of homogenous linear equations, i.e., a set; the aforementioned parameters for calculating the mutual information loss belong to the solution space of the aforementioned two input images; [symbol] can indicate mathematical expectation, P_{xz} is the joint probability distribution, Pₓ and P_{z} are the marginal probability distribution, θ is an initialization parameter of the mutual information estimating network, and n is a positive integer and can indicate a number of samples.

The larger the mutual information during training is, the more accurate the registration result is. Sup in the formula is a minimum upper bound, and increasing the minimum upper bound during the training is to maximize the mutual information. The aforementioned T may be understood as the mutual information estimating network model (including the parameters thereof). The mutual information may be estimated in combination with the formula, and therefore, the T here also has parameters that need to be updated. The formula and the T together constitute the mutual information loss.

At step 203, parameter updating is performed on the registering model and the mutual information estimating network model based on the mutual information loss to obtain a trained preset neural network model.

In the embodiments of the present disclosure, the neuron estimation mutual information is used as the similarity evaluation standard of the registered image and the reference image; that is, step 202 and step 203 may be repeatedly executed, the parameters of the registering model and the mutual information estimating network model are continuously updated to guide and complete the training of the two networks.

Optionally, parameter updating of the first threshold number of times is performed on the registering model according to the mutual information loss, and parameter updating of a second threshold number of times is performed on the mutual information estimating network model according to the mutual information loss to obtain the trained preset neural network model.

The image processing apparatus may store the first threshold number of times and the second threshold number of times, where the first threshold number of times and the second threshold number of times may be different, and the first threshold number of times may be greater than the second threshold number of times.

The first threshold number of times and the second threshold number of times involved in the aforementioned updating refer to epochs in the training of the neural network. One epoch may be understood as one forward delivery and one backward delivery of at least one training sample.

For example, independent parameter updating may be performed on the registering model and the mutual information estimating network model. For example, the first threshold number of times is 120, and the second threshold number of times is 50, that is, the mutual information estimating network model and the registering model may be updated together within first 50 epochs, and after the 50 epochs, the network parameters of the mutual information estimating network model are fixed, and only the registering model is updated until the updating of 120 epochs of the registering model is completed.

Optionally, parameter updating of a preset learning rate and a third threshold number of times is further performed on the preset neural network model according to the preset optimizer to obtain a final trained preset neural network model.

The algorithms used in the optimizer generally include an adaptive gradient (AdaGrad) optimization algorithm that relates to adjusting different learning rates for different parameters, updating a frequently changed parameter and a smaller step size, and updating a sparse parameter in a larger step size; and the RMSProp algorithm relates to adjusting the change of the learning rate in combination with the exponential moving average of the gradient square and may perform good convergence under the condition of a non-stationary target function.

The preset optimizer may be the optimizer having an ADAM, and combines the advantages of the AdaGrad and RMSProp algorithms. First moment estimation (i.e., the average value of gradients) and second moment estimation (i.e., the non-centralized variance of gradients) of gradients are comprehensively considered to calculate an update step size.

The third threshold number of times is the same as the first threshold number of times and the second threshold number of times, and refers to the epoch. The image processing apparatus or the preset optimizer may store the third threshold number of times and the preset learning rate so as to control updating. For example, the learning rate is 0.001, and the third threshold number of times is 300 epochs. The adjusting rule of the learning rate may be set, and the learning rate for parameter updating is adjusted according to the adjusting rule of the learning rate, such as the learning rate may be set to be respectively halved respectively at 40, 120 and 200 epochs.

After obtaining the trained preset neural network model, the image processing apparatus may execute some or all of the methods in the embodiments shown in FIG. 1. That is, the image to be registered may be registered with the reference image according to the preset neural network model to obtain the registration result.

Generally, in most technologies, the use of a non-parameterization method to estimate the mutual information (e.g., using a histogram) not only leads to large calculation amount but also does not support the backpropagation, and therefore, it is impossible to apply the technologies to the neural network. In the embodiments of the present disclosure, the neuron estimation mutual information is used to measure the similarity loss of the images. The trained preset neural network model may be used for image registration, and in particular, in the registration of any internal organ that may deform, may be used for performing deformable registration on follow-up images at different time points, so that the registration efficiency is high and the result is more accurate.

Generally, in some operations, it is required to perform one or more scans of different quality and speeds before or during the operation so as to obtain the medical image. However, medical image registration can usually only be performed after one or more scans have been performed. This does not meet a real-time requirement in the operation, so it usually takes extra time to judge the result of the operation. If it is discovered that the result of the operation is not ideal after the registration, follow-up operative treatment may be needed, which will waste time for doctors and patients and delay the treatment. Moreover, registration based on the preset neural network model in the embodiments of the present disclosure may be applied to real-time medical image registration in the operation. For example, real-time registration is performed in a tumor excision operation to determine whether the tumor is completely excised, and thus the timeliness is improved.

In the embodiments of the present disclosure, by obtaining a preset image to be registered and a preset reference image, and inputting the preset image to be registered and the reference image into the registering model to generate a deformable field, estimating, in a process of registering with the preset reference image based on the deformation field and the preset image to be registered, mutual information of the registered image and the preset reference image are estimated by means of the mutual information estimating network model to obtain the mutual information loss, performing parameter updating on the registering model and the mutual information estimating network model based on the mutual information loss to obtain a trained preset neural network model that can be applied to deformable registration, the precision and real-time performance of image registration are improved.

The solutions of the embodiments of the present disclosure are introduced above mainly from the perspective of a method-side execution process above. It can be understood that, to implement the foregoing functions, the image processing apparatus includes corresponding hardware structures and/or software modules for implementing the functions. A person skilled in the art should be easily aware that, in combination with the units and algorithm steps of the examples described in the embodiments disclosed in the specification, the present disclosure may be implemented by hardware or a combination of the hardware and computer software. Whether a certain function is implemented by hardware or hardware driven by computer software depends on particular applications and design constraint conditions of the technical solutions. A person skilled in the art may use different methods to implement the described functions for particular applications, but it should not be considered that the implementation goes beyond the scope of the present disclosure.

In the embodiments of the present disclosure, the image processing apparatus may be divided into functional modules according to the foregoing method examples. For example, each functional module may be obtained according to each function. Two or more functions may also be integrated into one processing module. The integrated module may be implemented in the form of hardware or in the form of a software functional module. It should be noted that the division of modules in the embodiments of the present disclosure is exemplary, and is only division of logical functions. Other division manners may be available in actual implementations.

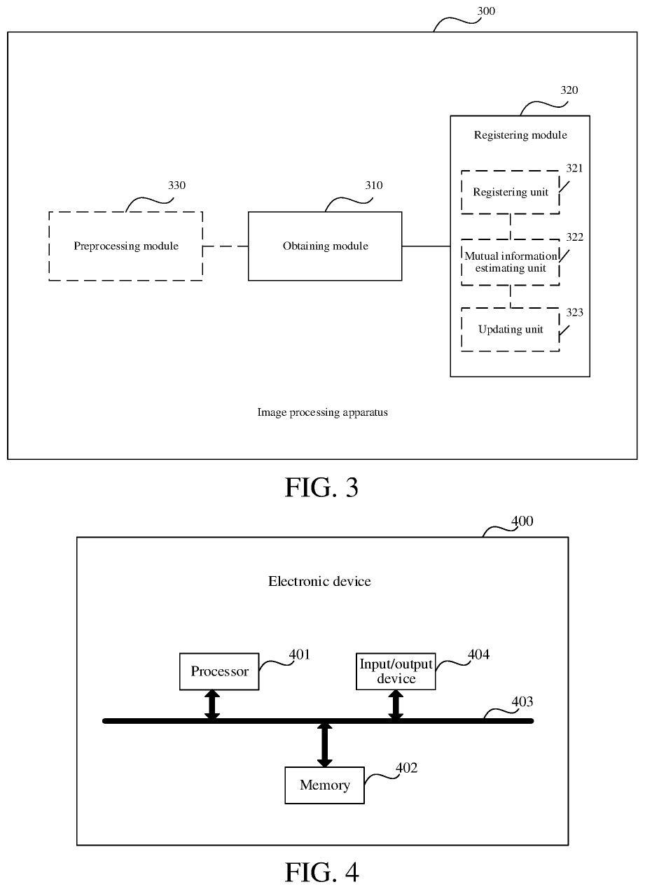

Please refer to FIG. 3. FIG. 3 is a schematic structural diagram of an image processing apparatus according to embodiments of the present disclosure. As shown in FIG. 3, the image processing apparatus 300 includes an obtaining module 310 and a registering module 320, where

the obtaining module 310 is configured to obtain an image to be registered and a reference image used for registration;

the registering module 320 is configured to input the image to be registered and the reference image into a preset neural network model, where the preset neural network model is obtained by training based on the mutual information loss of the preset image to be registered and the preset reference image; and

the registering module 320 is further configured to register the image to be registered with the reference image to obtain a registration result.

Optionally, the image processing apparatus 300 further includes a preprocessing module 330 configured to obtain an original image to be registered and an original reference image, and perform image normalization processing on the original image to be registered and the original reference image to obtain the image to be registered and the reference image that meet the target parameters.

Optionally, the preprocessing module 330 is specifically configured to:

convert the original image to be registered into the image to be registered within a preset gray value range and with a preset image size; and

convert the original reference image into the reference image within the preset grey-scale value range and with the preset image size.

Optionally, the preset neural network model includes a registering model and a mutual information estimating network model, and the registering module 320 includes a registering unit 321, a mutual information estimating unit 322, and a updating unit 323, where:

the registering unit 321 is configured to obtain the preset image to be registered and the preset reference image, and input the preset image to be registered and the preset reference image into the registering model to generate a deformable field;

the mutual information estimating unit 322 is configured to estimate, in a process of registering with the preset reference image by the registering module based on the deformation field and the preset image to be registered, mutual information of a registered image and the preset reference image by means of the mutual information estimating network model to obtain mutual information loss; and

the updating unit 323 is configured to perform parameter updating on the registering model and the mutual information estimating network model based on the mutual information loss to obtain a trained preset neural network model.

Optionally, the mutual information estimating unit 322 is specifically configured to:

obtain joint probability distribution and marginal probability distribution by means of the mutual information estimating network model based on the registered image and the preset reference image; and

calculate the mutual information loss according to the joint probability distribution and the marginal probability distribution.

Optionally, the updating unit 323 is specifically configured to:

perform parameter updating of a first threshold number of times on the registering model based on the mutual information loss, and perform parameter updating of a second threshold number of times on the mutual information estimating network model based on the mutual information loss to obtain a trained preset neural network model.

Optionally, the updating unit 323 is further configured to perform parameter updating of a preset learning rate and a third threshold number of times on the preset neural network model based on a preset optimizer.

Optionally, the preprocessing module 330 is further configured to:

perform image normalization processing on the preset image to be registered and the preset reference image to obtain the preset image to be registered and the preset reference image that meet preset training parameters.

The registering module is further configured to input the preset image to be registered and the preset reference image that meet the preset training parameters into the registering model to generate the deformable field.

The image processing apparatus 300 in the embodiments shown in FIG. 3 may implement some or all of the methods in the embodiments shown in FIG. 1 and/or FIG. 2.

The image processing apparatus 300 shown in FIG. 3 is implemented. The image processing apparatus 300 may obtain an image to be registered and a reference image used for registration, input the image to be registered and the reference image into a preset neural network model, where the preset neural network model is obtained by training based on mutual information loss of the preset image to be registered and the preset reference image, and register the image to be registered with the reference image according to the preset neural network model to obtain a registration result. The precision and real-time performance of image registration can be improved.

In some embodiments, the functions provided by or the modules included in the apparatus provided by the embodiments of the present disclosure may be used for implementing the methods described in the foregoing method embodiments. For the specific implementations of the methods, reference may be made to the descriptions in the method embodiments above. For the purpose of brevity, detailed are not described herein again.

Please refer to FIG. 4. FIG. 4 is a schematic structural diagram of an electronic device according to embodiments of the present disclosure. As shown in FIG. 4, the electronic device 400 includes a processor 401 and a memory 402. The electronic device 400 may further include a bus 403. The processor 401 and the memory 402 may be interconnected by means of the bus 403. The bus 403 may be a Peripheral Component Interconnect (PCI) bus or an Extended Industry Standard Architecture (EISA) bus and the like. The bus 403 may be classified into an address bus, a data bus, a control bus, and the like. For ease of representation, only one bold line is used for representation in FIG. 4. However, this does not indicate that there is only one bus or only one type of bus. The electronic device 400 may further include an input/output device 404 that may include a display screen, such as a liquid crystal display screen. The memory 402 is configured to store one or more programs including instructions. The processor 401 is configured to invoke the instructions stored in the memory 402 to implement some or all of the steps of the methods mentioned in the embodiments in FIGS. 1 and 2. The foregoing processor 401 may correspondingly implement the functions of the modules in the image processing apparatus 300 in FIG. 3.

The image processing apparatus 400 shown in FIG. 4 is implemented. The image processing apparatus 400 may obtain an image to be registered and a reference image used for registration, input the image to be registered and the reference image into a preset neural network model, where the preset neural network model is obtained by training based on mutual information loss of the preset image to be registered and the preset reference image, and register the image to be registered with the reference image based on the preset neural network model to obtain a registration result. The precision and real-time performance of image registration can be improved.

The embodiments of the present disclosure further provide a computer readable storage medium, where the computer readable storage medium stores a computer program for electronic data interchange, and the computer program enables the computer to implement some or all of the steps of any one image processing method recited in the foregoing method embodiments.

The embodiments of the present disclosure further provide a computer program product, including a computer readable code, where when the computer readable code runs in a device, a processor in the device executes instructions for implementing the image processing method provided by any one of the foregoing embodiments.

It should be noted that, for ease of description, the foregoing method embodiments are expressed as a series of action combinations. However, a person skilled in the art should know that the present disclosure is not limited by the order of actions described because certain steps may be executed in any other order or simultaneously according to the present disclosure. Furthermore, a person skilled in the art should also know that the embodiments described in the specification are preferred embodiments, and the involved actions and modules are not necessarily required by the present disclosure.

In the aforementioned embodiments, description of the embodiments all have their own focuses, and for portions that are not described in detail in one embodiment, refer to the related description in other embodiments.

It should be understood that the disclosed apparatus in the embodiments provided in the present disclosure may be implemented by means of other modes. For example, the apparatus embodiments described above are merely exemplary. For example, the division of modules (or units) is merely logical function division and may be other division in actual implementation. For example, a plurality of modules or components may be combined or integrated into another system, or some features may be ignored or not performed. In addition, the displayed or discussed mutual couplings or direct couplings or communication connections may be implemented by means of some interfaces. The indirect couplings or communication connections between the apparatuses or modules may be implemented in electronic, mechanical, or other forms.

The modules described as separate parts may or may not be physically separate, and the parts displayed as modules may or may not be physical modules, may be located at one position, or may be distributed on a plurality of network modules. Some or all of the modules may be selected according to actual needs to achieve the objectives of the solutions of the embodiments.

In addition, functional modules in the embodiments of the present disclosure may be integrated into one processing module, or each of the modules may exist alone physically, or two or more modules may be integrated into one module. The integrated module may be implemented in a form of hardware and may also be implemented in a form of a software functional module.

If the integrated module is implemented in the form of the software functional module and sold or used as an independent product, it may be stored in one computer readable memory. Based on such an understanding, the technical solutions of the present disclosure essentially, or some of the technical solutions contributing to the prior art, or all or some of the technical solutions may be implemented in the form of a software product. A computer software product is stored in one memory and includes several instructions used for making a computer device (which may be a personal computer, a server or a network device and the like) execute all or some of the steps of the methods described in the embodiments of the present disclosure. Moreover, the foregoing memory includes: any medium that can store program codes, such as a USB flash drive, a Read-Only Memory (ROM), a Random Access Memory (RAM), a mobile hard disk, a magnetic disk, or an optical disk.

A person of ordinary skilled in the art can understood that all or some of the steps of the methods in the foregoing embodiments may be implemented by instructing related hardware by means of the programs. The programs may be stored in one computer readable memory, and the memory may include a flash memory, an ROM, an RAM, a magnetic disk or an optical disk.

The embodiments of the present disclosure are introduced in details above, and the principles and implementations of the present disclosure are explained by applying specific examples. However, the descriptions of the embodiments above are merely used for helping understand the method and core concept of the present disclosure. In addition, a person of ordinary skilled in the art may make modifications to the specific implementations and application range according to the concept of the present disclosure. Hence, the content in the present specification should not be understood to limit the present disclosure.

What is claimed is:

1. An image processing method, comprising:
obtaining an image to be registered and a reference image used for registration;
inputting the image to be registered and the reference image into a preset neural network model, wherein the preset neural network model is obtained by training based on mutual information loss of a preset image to be registered and a preset reference image; and
registering the image to be registered with the reference image based on the preset neural network model to obtain a registration result.

2. The image processing method according to claim 1, wherein before obtaining the image to be registered and the reference image used for registration, the method further comprises: obtaining an original image to be registered and an original reference image, and performing image normalization processing on the original image to be registered and the original reference image to obtain the image to be registered and the reference image that meet target parameters.

3. The image processing method according to claim 2, wherein performing image normalization processing on the original image to be registered and the original reference image to obtain the image to be registered and the reference image that meet the target parameters comprises:
converting the original image to be registered into the image to be registered within a preset gray value range and with a preset image size; and
converting the original reference image into the reference image within the preset gray value range and with the preset image size.

4. The image processing method according to claim 1, wherein the preset neural network model comprises a registering model and a mutual information estimating network model, and a training process of the preset neural network model comprises:
obtaining the preset image to be registered and the preset reference image, and inputting the preset image to be registered and the preset reference image into the registering model to generate a deformable field;
estimating, in a process of registering with the preset reference image based on the deformable field and the preset image to be registered, mutual information of a registered image and the preset reference image by means of the mutual information estimating network model to obtain the mutual information loss; and
performing parameter updating on the registering model and the mutual information estimating network model based on the mutual information loss to obtain a trained preset neural network model.

5. The image processing method according to claim 4, wherein estimating the mutual information of the registered image and the preset reference image by means of the mutual information estimating network model to obtain the mutual information loss comprises:
obtaining joint probability distribution and marginal probability distribution by means of the mutual information estimating network model based on the registered image and the preset reference image; and
calculating the mutual information loss according to the joint probability distribution and the marginal probability distribution.

6. The image processing method according to claim 4, wherein performing parameter updating on the registering model and the mutual information estimating network model based on the mutual information loss to obtain the trained preset neural network model comprises:
performing parameter updating of a first threshold number of times on the registering model based on the mutual information loss, and performing parameter updating of a second threshold number of times on the mutual information estimating network model based on the mutual information loss, to obtain the trained preset neural network model.

7. The image processing method according to claim 6, further comprising:
performing parameter updating of a preset learning rate and a third threshold number of times on the preset neural network model based on a preset optimizer.

8. The image processing method according to claim 4, wherein after obtaining the preset image to be registered and the preset reference image, the method further comprises:
performing image normalization processing on the preset image to be registered and the preset reference image to obtain the preset image to be registered and the preset reference image that meet preset training parameters; and
inputting the preset image to be registered and the preset reference image into the registering model to generate the deformable field comprises:
inputting the preset image to be registered and the preset reference image that meet the preset training parameters into the registering model to generate the deformable field.

9. An image processing apparatus, comprising:
a processor; and
a memory storing one or more processor-executable programs which, when executed by the processor, cause the processor to:
obtain an image to be registered and a reference image used for registration;
input the image to be registered and the reference image into a preset neural network model, wherein the preset neural network model is obtained by training based on mutual information loss of a preset image to be registered and a preset reference image; and
register the image to be registered with the reference image based on the preset neural network model to obtain a registration result.

10. The image processing apparatus according to claim 9, wherein before obtaining the image to be registered and the reference image used for registration, the processor is further caused to obtain an original image to be registered and an original reference image, and perform image normalization processing on the original image to be registered and the original reference image to obtain the image to be registered and the reference image that meet target parameters.

11. The image processing apparatus according to claim 10, wherein performing image normalization processing on the original image to be registered and the original reference image to obtain the image to be registered and the reference image that meet the target parameters comprises:
converting the original image to be registered into the image to be registered within a preset gray value range and with a preset image size; and
converting the original reference image into the reference image within the preset gray value range and with the preset image size.

12. The image processing apparatus according to claim 9, wherein the preset neural network model comprises a registering model and a mutual information estimating network model, and the processor is further caused to train the preset neural network model by:
obtaining the preset image to be registered and the preset reference image, and inputting the preset image to be registered and the preset reference image into the registering model to generate a deformable field;
estimating, in a process of registering with the preset reference image by the registering module based on the deformable field and the preset image to be registered, mutual information of a registered image and the preset reference image by means of the mutual information estimating network model to obtain mutual information loss; and
performing parameter updating on the registering model and the mutual information estimating network model based on the mutual information loss to obtain a trained preset neural network model.

13. The image processing apparatus according to claim 12, wherein estimating the mutual information of the registered image and the preset reference image by means of the mutual information estimating network model to obtain the mutual information loss comprises:
obtaining joint probability distribution and marginal probability distribution by means of the mutual information estimating network model based on the registered image and the preset reference image; and
calculating the mutual information loss according to the joint probability distribution and the marginal probability distribution.

14. The image processing apparatus according to claim 12, wherein performing parameter updating on the registering model and the mutual information estimating network model based on the mutual information loss to obtain the trained preset neural network model comprises:
performing parameter updating of a first threshold number of times on the registering model based on the mutual information loss, and performing parameter updating of a second threshold number of times on the mutual information estimating network model based on the mutual information loss, to obtain the trained preset neural network model.

15. The image processing apparatus according to claim 14, wherein the processor is further caused to perform parameter updating of a preset learning rate and a third threshold number of times on the preset neural network model based on a preset optimizer.

16. The image processing apparatus according to claim 12, wherein after obtaining the preset image to be registered and the preset reference image, the processor is further caused to:
performing image normalization processing on the preset image to be registered and the preset reference image to obtain the preset image to be registered and the preset reference image that meet preset training parameters; and
inputting the preset image to be registered and the preset reference image that meet the preset training parameters into the registering model to generate the deformable field.

17. A non-transitory computer readable storage medium, wherein the computer readable storage medium is used for storing a computer program for electronic data interchange, and the computer program enables a computer to execute an image processing method, comprising:
obtaining an image to be registered and a reference image used for registration;
inputting the image to be registered and the reference image into a preset neural network model, wherein the preset neural network model is obtained by training based on mutual information loss of a preset image to be registered and a preset reference image; and
registering the image to be registered with the reference image based on the preset neural network model to obtain a registration result.

18. The medium according to claim 17, wherein before obtaining the image to be registered and the reference image used for registration, the method further comprises:
obtaining an original image to be registered and an original reference image, and performing image normalization processing on the original image to be registered and the original reference image to obtain the image to be registered and the reference image that meet target parameters.

19. The medium according to claim 18, wherein performing image normalization processing on the original image to be registered and the original reference image to obtain the image to be registered and the reference image that meet the target parameters comprises:
converting the original image to be registered into the image to be registered within a preset gray value range and with a preset image size; and
converting the original reference image into the reference image within the preset gray value range and with the preset image size.

20. The medium according to claim 17, wherein the preset neural network model comprises a registering model and a mutual information estimating network model, and a training process of the preset neural network model comprises:
obtaining the preset image to be registered and the preset reference image, and inputting the preset image to be registered and the preset reference image into the registering model to generate a deformable field;
estimating, in a process of registering with the preset reference image based on the deformable field and the preset image to be registered, mutual information of a registered image and the preset reference image by means of the mutual information estimating network model to obtain the mutual information loss; and
performing parameter updating on the registering model and the mutual information estimating network model based on the mutual information loss to obtain a trained preset neural network model.
